(19) United States
(12) Patent Application Publication
(10) Pub. No.: US 2004/0117014 A1
(43) Pub. Date: Jun. 17, 2004

(54) Prosthetic implant assembly

(76) Inventor: Julian Bryant, Leeds (GB)

Correspondence Address: MADSON & METCALF, GATEWAY TOWER WEST, SUITE 900, 15 WEST SOUTH TEMPLE, SALT LAKE CITY, UT, 84101

(21) Appl. No.: 10/720,036

(22) Filed: Nov. 21, 2003

(30) Foreign Application Priority Data
Nov. 21, 2002 (GB) 0227161.7

(51) Int. Cl. A61F 2/08

(52) U.S. Cl. 623/13.12; 623/13.14; 623/20.14

(57) ABSTRACT

A prosthetic implant kit for implantation in a bone joint between first and second bones, such as the knee joint between a femur and a tibia, such joint having bone tunnels formed in each of the first and second bones, and the implant assembly being intended to be introduced into the bone tunnels and to be fixated therein, and implant kit assembly comprising:

a prosthetic implant e.g. a ligament to be fitted in the bone tunnels;

a degradable adhesive and/or filler material to be introduced into the bone tunnel to fill the space available between the implant and the wall of the bone tunnel, such maternal being of such a nature that it is capable of setting in order temporarily to anchor the implant in position, but which degrades over time so as to allow natural boney ingrowth to take over the anchoring of the implant; and

a tunnel profiler to form an enlarged entry cavity to the bone tunnels to be filled with the material and improve the temporary fixation of the implant.

This invention relates to a prosthetic implant assembly which is intended to be installed in a bone joint between first and second bones, such as the knee joint between a femur and a tibia, in which bone tunnels are formed in each of the first and second bones, and a prosthetic implant is taken through the bone tunnels and is subsequently anchored therein.

### BACKGROUND TO INVENTION

The most common ligament injury is the knee anterior cruciate ligament (ACL), which does not heal and is commonly replaced with an autologous graft or an artificial ligament. However most permanent ligament prostheses still perform poorly in the long-term due to pain, sterile effusions, arthritis and mechanical breakdown of the synthetic polymers. Wear debris particles may also accelerate damage to surrounding tissues. The method of attachment of the ligament prosthesis onto the bone is another issue affecting the success of the implant.

The most fundamental and most common outcome of torn anterior cruciate ligament is a knee that is looser than it was before the injury. The normal anterior cruciate ligament guides and restrains the motion of the knee within a very small 2-5 mm range Unfortunately, tearing is usually an “all or none” phenomenon except in very rare circumstances when only part of the ligament actually tears.

Many patients, then, experience a sense of instability in their knees after ACL injury, particularly when the knee is subjected to the higher loads of jumping, landing, planting and pivoting, or rapid acceleration or deceleration Under those circumstances, but rarely with the lesser loads of everyday activities, the knee will “give way”, sometimes very painfully and accompanied by a fall. If not corrected by ACL reconstruction the patient develops an abnormal loading pattern that protects the knee but puts enormous strain on other joints in the body.

Currently the techniques for reconstructing the ACL consist of creating tibial and femoral tunnels for the natural or artificial ligament to pass through and a mechanical fixation device to hold them in place under the correct tension. In order to restore the stability of the knee the correct tensioning must be established. At present endobuttons, staples, interference screws and cross pin techniques are utilised to fix the ligament. The common mode of failure of these devices is their loosening due to bone degradation around the implant This leads to slackening of the ligament and the patient is back to square one with a dysfunctional knee. Another significant disadvantage with the current ACL fixation method is that in some patients, (especially thin patients) the hardware may become prominent causing local irritation and pain and may need to be removed.

### SUMMARY OF INVENTION

The present invention has been developed with a view to provide a new ACL fixation technique, and which seeks to eliminate many of the most common problems encountered with existing ACL fixation techniques.

According to one aspect of the invention there is provided a prosthetic implant assembly for implantation in a bone joint between first and second bones, such as the knee joint between a femur and a tibia, said bone joint having bone tunnels formed in each of the first and second bones, and the implant assembly being intended to be introduced into the bone tunnels and to be fixated therein, in which the prosthetic implant assembly comprises:

a prosthetic ligament to be fitted in at least one of the bone tunnels; and,

a degradable adhesive and/or filler material (which may provide permanent or temporary fixation) to be introduced into said one bone tunnel to fill the space available between at least part of the prosthetic ligament and the wall of the bone tunnel, said material being of such a nature that (a) it is capable of setting in order temporarily to anchor said part of the ligament in position, and (b) it degrades over time such as to allow natural boney ingrowth to take over the anchoring of the prosthetic ligament.

According to a second aspect of the invention there is provided a prosthetic implant kit for implantation in a bone tunnel in a bone joint, said kit comprising:

a prosthetic implant to be fitted in said bone tunnel;

a degradable adhesive and/or filler material to be introduced into said bone tunnel to fill the space available between at least part of the prosthetic implant and the wall of the bone tunnel, said material being of such a nature that (a) it is capable of setting in order temporarily to anchor said part of the implant in position, and (b) it degrades over time such as to allow natural boney ingrowth to take over the anchoring of the prosthetic implant; and,

a tunnel profiler to form an enlarged entry cavity to the bone tunnel to be filled with said material, and improve the temporary fixation of the implant.

According to a third aspect of the invention there is provided a method of installing a prosthetic implant in a bone joint between first and second bones, such as the knee joint between a femur and a tibia, in which:

bone tunnels are formed in each of the first and second bones;

a prosthetic implant is taken through the bone tunnels; and, a degradable adhesive and/or filler material is introduced into at least part of one of the tunnels and which is caused or allowed to set in order temporarily to anchor part of the implant in position, such material being of such a type as to degrade over time such as to allow natural boney ingrowth to take over the anchoring of the implant.

Therefore, the invention utilises an adhesive and/or filler material which is of such a nature or type that it is capable of setting or curing, to provide temporary fixation or anchoring of at least part of the implant in a bone tunnel, but the material degrades over time and then allows natural boney tissue ingrowth to take over (from the initial temporary fixation) and form a permanent or semi-permanent fixation or anchoring of the implant.

As is disclosed in subsequent preferred examples, currently available “bone cements” may be particularly useful for carrying out the invention.

In addition to the provision of a tunnel profiler, according to the third aspect of the invention, a fixation device may also be provided. The tunnel profiler can be used to produce a shaped cavity at the end of the bone tunnel, which would aid fixation between the bone cement and surrounding bone. A fixation device may also be used to aid fixation between the implant e.g. a ligament and bone cement.

Preferred embodiments of the invention will be described in detail, by way of example only, with reference to the accompanying drawings.

### BRIEF DESCRIPTION OF DRAWINGS

FIG. 1 is a perspective and diagrammatic illustration of a single strand or looped structure of a prosthetic implant for use in carrying out the invention;

FIG. 2 illustrates a knotted ligament, to provide an implant with improved means of anchoring in situ;

FIG. 3 is a perspective illustration of a further example of prosthetic implant for use in carrying out the invention;

FIG. 4 is a diagrammatic illustration of a tunnel profiler and a cavity it produces, for carrying out a preferred development of the invention;

FIG. 5 illustrates alternative basic profiles for the tunnel profiler;

FIG. 6 illustrates further examples of profile of a profiler;

FIG. 7 shows further examples of screw thread features to be provided on a tunnel profiler;

FIG. 8 illustrates examples of open and closed screw threads of a tunnel profiler;

FIG. 9 shows still further examples of fins, barbs, stepped and flush profiles, to be provided on a profiler;

FIG. 10 illustrates successive surgical technique steps involved in producing a profiled hole;

FIG. 11 shows a preferred example of a pouched ligament for use in carrying out the invention;

FIG. 12 illustrates spheres attached to a prosthetic ligament to provide improved anchoring;

FIG. 13 illustrates toggles and crimped designs attached to the ligament;

FIG. 14 illustrates surgical technique to “cement” i.e. adhere the ligament in a cavity, and,

FIG. 15 illustrates a still further example according to the invention.

Preferred examples of prosthetic implant assemblies or kits, and methods of installation thereof, will now be described in detail with reference to the accompanying drawings.

### DESCRIPTION OF PREFERRED EMBODIMENTS

Ligament

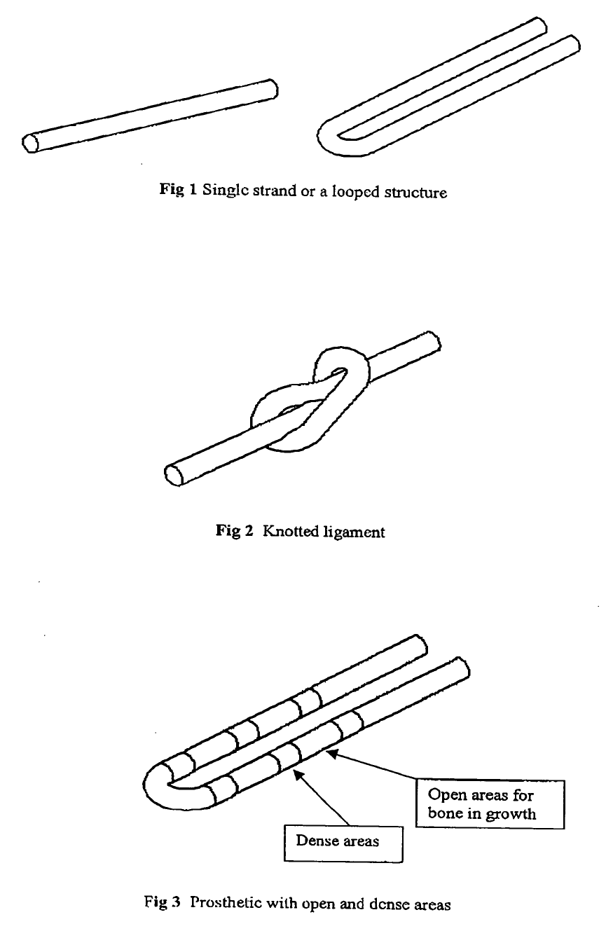

The ligament may be an allograft, autograft or a prosthesis. This ligament may be a single strand or a looped structure. Multiple stands or loops could be used (FIG. 1).

A positive interlock between the ligament and bone cement can be achieved to aid fixation. This could be produced by the ligament having a rough surface. Alternatively the ligament could be knotted once, or in several placed along its length such that the bone cement would encapsulate these features (FIG. 2). In the case of the prosthetic it may consist of a series of integral “open” and “dense” areas (FIG. 3). The open areas would allow a greater amount of cement penetration and hence provide a greater interlock.

Bone Cement

The cement may act as an adhesive to attach the ligament to the adjacent bone. Alternatively it may act as a filler material, fixing the ligament to the bone by mechanical interlock due to the rough surfaces.

The cement should provide immediate structural support but should not allow the bone to regenerate and eventually fail through fatigue and degradation causing device loosening and bone loss. Lower exotherm, improved fracture toughness, biodegradable bone cements with good tissue compatibility are now available. The material needs to allow normal bone healing by degrading at a rate tuned to the healing process and not act as a barrier to bone regeneration.

Inject able formulations for use in minimally invasive procedures are also desirable in our design. The cement should bond chemically to bone and prostheses. It should preferably be bioactive and stimulate bone growth and be capable of acting as a suitable medium for the release of water-soluble antibiotics.

The cement may be a standard material that is available “off the shelf”, or a special formula for this application. The fixation matter used in our design could be a bone cement, a glue or a polymer mix. The cement may be permanent (PMMA) or biodegradable (PLLA, PGA PLLA-HA etc). It would preferably be of a compound that does not generate heat during setting. It may for instance be possible to set such a material using ultraviolet light like those used for dental fillings. It could be solid or porous to allow bone ingrowth. It may have additional properties or additives to accelerate bone in growth or prevent infection. It would be advantageous if the cement had a short cure time, to minimise the time required to perform the operation.

There are four main types of bone cement

PMMA based bone cements

Calcium Phosphate based bone cements

Glass based bone cements

Inorganic bone cements

The subsequent table shows a few of the commercially available bone cements that could be incorporated into our design

PMMA Based

PMMA

PBMMA

B-Palacos 50

B-Palacos 70

Palacos gamma 1

Palacos gamma 2

Palacos R

Copal Palacos R

Refobacin Palacos R

Simplex P

Zimmer regular

Zimmer LVC

Osteobond

C-CMW 1

B-CMW 1

CMW 3

B-CMW 1

B-Surg Simp

B-270

B 1200

Calcium Based

dicalcium phosphate dihydrate (DCPD

tetracalcium phosphate (TTCP)

Calcibon (calcium phosphate)

Bone source

Calcium Phosphate

Calcium Sulphate

Beta Tri Calcium P

Hydroxy apatite

Glass Based

Glass Polyalkenoate

Glass ionomer Cements

Other

Zyment

Injectable Inorganic cements

Palamed

Palamed G

Osteopal

Osteopal G

Strontium-containing hydroxyapatite powder and D-GMA resin

These bone cements may have antibiotics incorporated. (e.g. Refobacin Palacos R). The main antibiotic bone cements currently on the market incorporate the Gentamicin antibiotic.

To improve mechanical properties fibres may be integrated into the cement. The fibres currently used for this purpose are Titanium fibres, polyethylene and glass fibres into PMMA based bone cements.

To lock the ligament in place pockets in the ligament could be designed so that they could hold solid matter that would prevent the ligament slipping from its desired location and maintain the ligaments tension. This could be done by coating the inner side of the pocket section of the ligament with a catalyst or similar compound. When the bone cement is injected the catalyst type compound would initiate the polymerisation of the bone cement and cause it to expand. The expanded matter would be too large to fit through the profiled tunnel and would therefore provide the required interlock. Another method that could be utilised would be to encapsulate bone cement and a catalyst in separate capsules within the ligament pockets. When in situ the capsules could be mechanically deformed or chemically degraded to initiate the polymerisation of the bone cement and expand the ligament. Table 1 confers the different combination of materials that could be utilized to solve the problem.

Tunnel Profiler

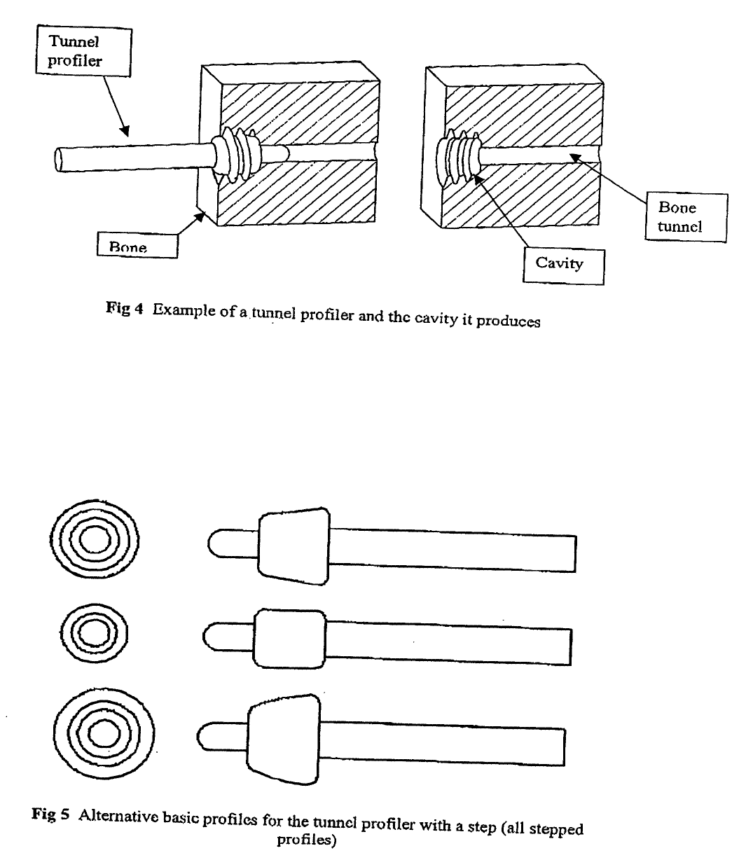

The bone tunnel may be profiled to create a cavity that can be filled with cement to provide a positive engagement (FIG. 4). This will aid fixation and help prevent movement between the bone cement and surrounding bone. This would increase the pull out strength of the device. Since this limits micro motion direct bone in growth may be possible (not fibrous tissue).

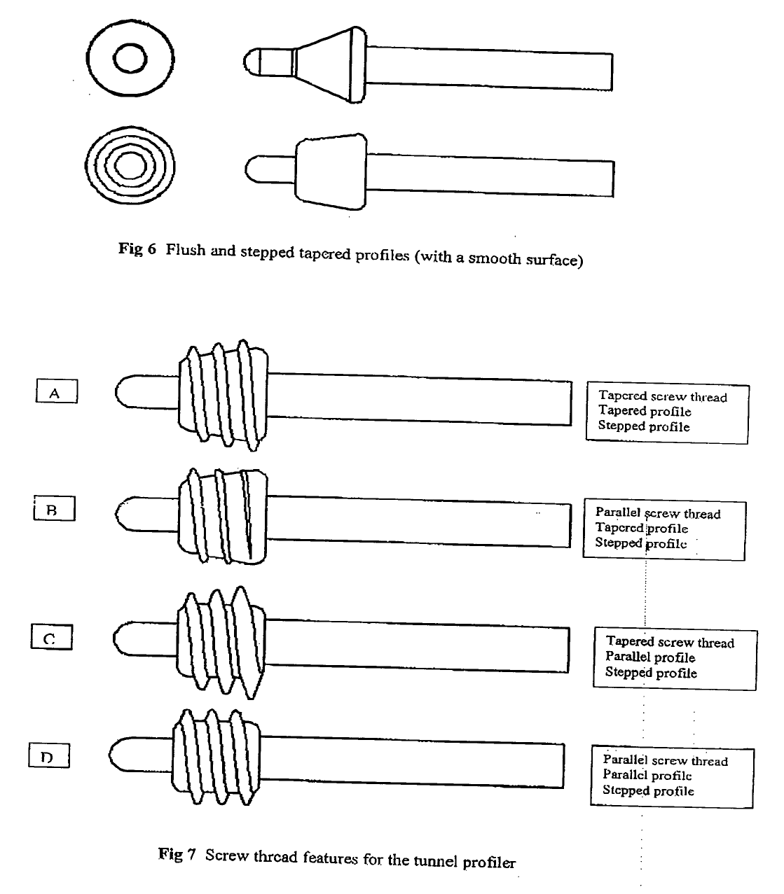

The basic profile of the device may be parallel sided, tapered, convex or concave (FIG. 5). The profile may also be flush with the tunnel diameter or be stepped out (FIG. 6). Such a cavity may be created by an instrument that is used like a dilator (punch action) or a tap (screw action). Such instruments may have a smooth surface or a roughened surface like a rasp, to aid producing the cavity.

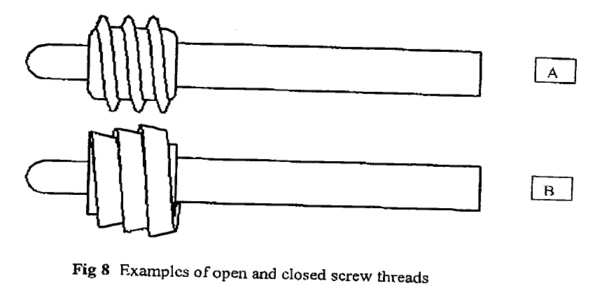

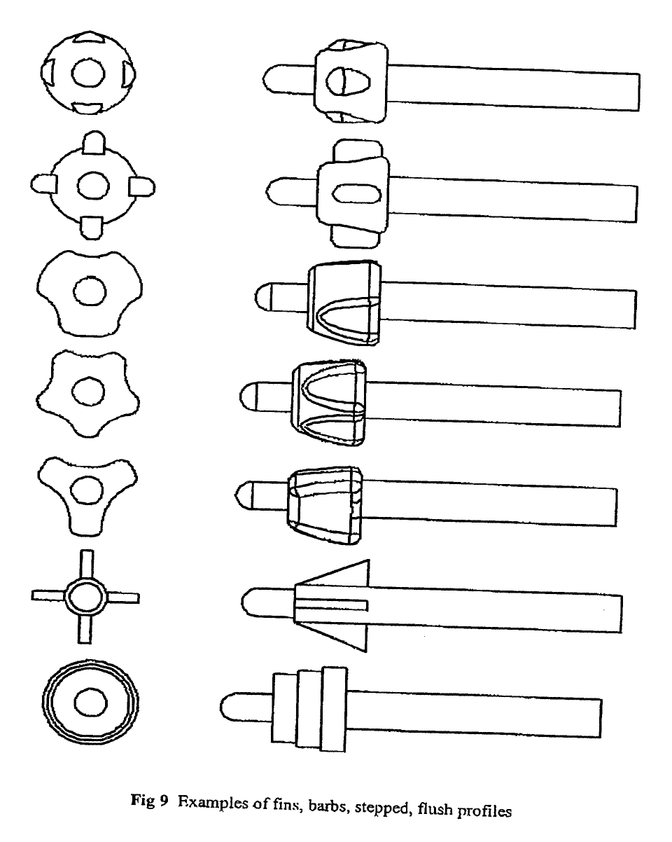

Features can also be incorporated into the design of the tunnel profiler to produce a complex shaped cavity to further aid fixation between the bone cement and bone. The instrument and therefore cavity may have screw threads, fins, barbs, taper, step in diameter, or a combination of these or similar shaped features. Examples of a screw thread are given in FIG. 7. The screw thread may have an “open” appearance and have a standard bone screw profile, or it may use a thread that has a thread depth and pitch that is optimised to provide a high fixation strength (FIG. 8a). Alternatively, the screw thread may have a “closed” appearance to provide a helical stepped profile (FIG. 8b).

Examples of features such as fins, barbs etc are given in FIG. 9.

The shape of the features on the instrument will determine which type of action will be employed when it is used. For example, a punch action if fins are incorporated or a screw action if a thread is integral to the design.

More than one instrument may be used to produce a complex profiled hole. For instance, a thread profiler may be used with a screw action to produce a profile to aid pullout strength of the cement block. A dilator with fins could follow this, which would prevent the cement from rotating. Having a profiled tunnel will establish a secure fixation when the bone cement polymerises in situ creating an object that is to great to fit out of the tunnel hence providing the critical tension.

The instrument can be designed to be used in a number of different surgical techniques. If for example an 8 mm tunnel is required with a profiled hole, the following alternative designs of tunnel profiler can be used

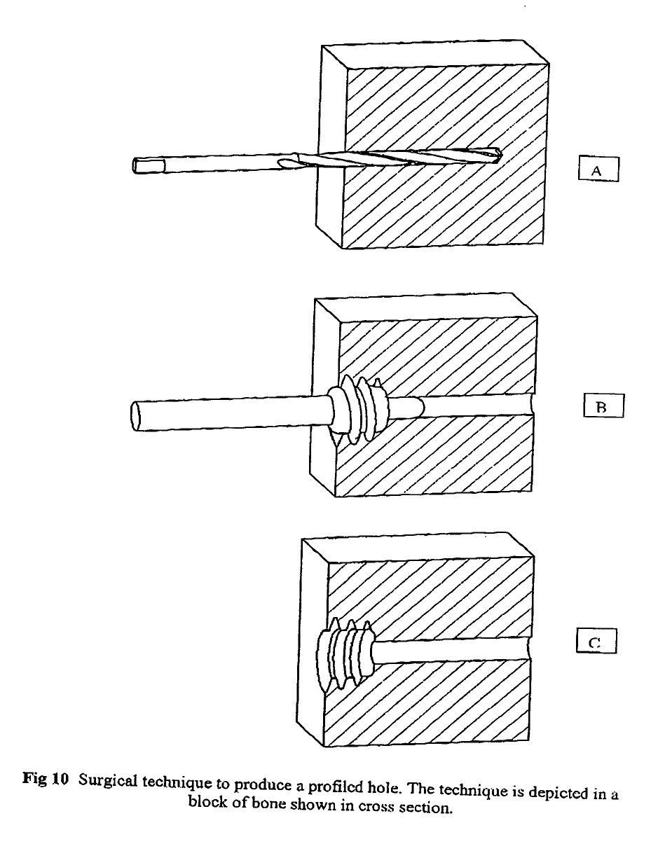

A standard medical drill with a diameter of 8 mm can be used to form the bone tunnel (FIG. 10a). The nose of the tunnel profiler instrument would therefore be slightly smaller than 8 mm in diameter (for example 7.45 mm) so that it can be inserted into the bone tunnel and used to guide the instrument during use (FIG. 10b), to produce the cavity (FIG. 10c).

A drill with a smaller diameter, say 7 mm, may be used to form the bone tunnel. The nose of the tunnel profiler may be 8 mm in diameter and elongated and used to dilate the bone tunnel to the correct diameter at the same time as the cavity is produced.

If the tunnel profiler does not have any additional features it can be incorporated into the drill such that a single instrument can be used to produce the bone tunnel and cavity in a single operation.

Fixation device

The fixation device would provide additional interlock between the ligament and the bone cement. This device may be internal or external to the ligament. This may also be integral with the ligament or a separate component.

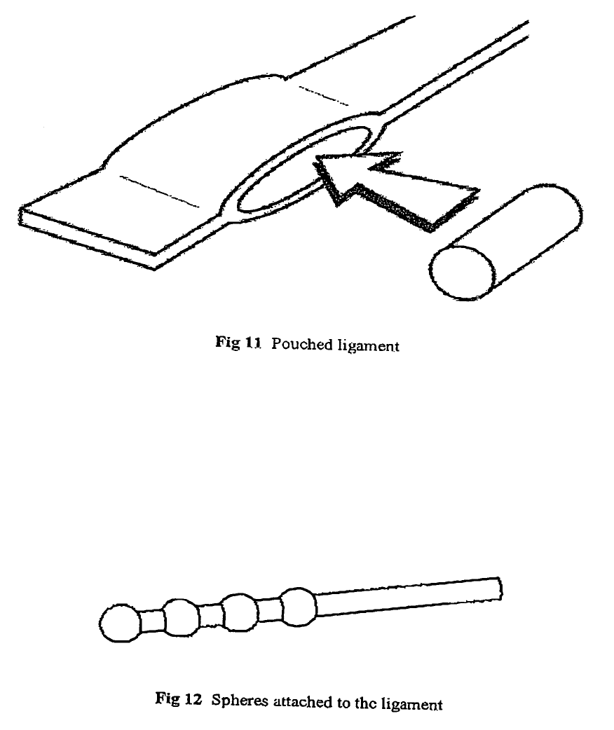

An example of an internal device would be a component that could be placed inside a pouch that is part of the prosthetic (FIG. 11). The device may be a simple shape such as a sphere. These spheres could be the same size or get progressively larger or smaller. The internal device may be more complex, for instance a tapered cylinder that could have additional fins or barbs to aid fixation with the bone cement and ligament.

An integral fixation device could be accomplished in the case of the prosthetic by having spherical blobs attached directly to the prosthetic (FIG. 12). These spheres or similar shaped device may be sutured, glued, or moulded to the ligament. Due to the ligaments having pockets bone cement or a similar material could be injected to fill the pockets so they create a fixation mechanism.

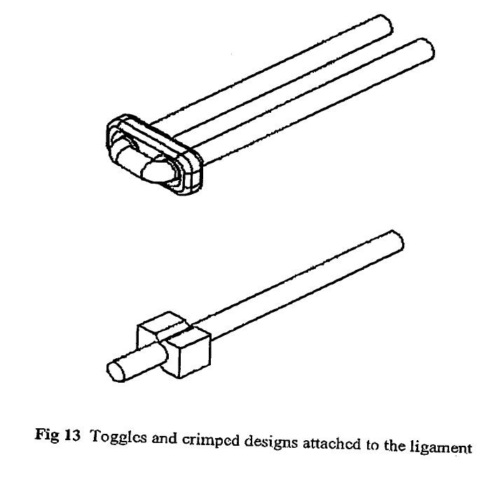

Various designs of toggle could also be used over a looped ligament (FIG. 13). A block could be used on a straight length of ligament. This could be crimped onto the ligament, or it could be attached with a grub screw. These components could also incorporate barbs of fins to prevent them moving into the bone cement when under load from the ligament.

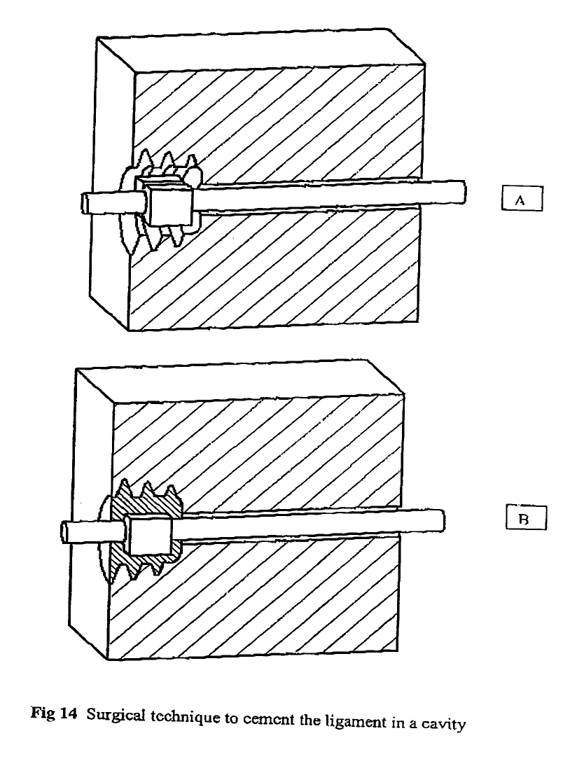

Once the toggle is correctly located in the bone tunnel (FIG. 14a), the correct tension is applied to the ligament and the bone cement is used to fill the space (FIG. 14b)

Such fixation devices may be manufactured from a metal, plastic or ceramic. It may be either permanent or bioabsorbable. Examples of metal devices would be titanium, cobalt chrome or stainless steel. Non resorbable polymers would include materials such as polyethylene, polyacetal, acetal and polypropylene. Examples of resorbable polymers include Trimethylene carbonate (TMC), Polyglycolic Acid (PGA), poly-L-lactic acid (PLLA), Poly D, L Lactic Acid (PDLLA), Poly D, L Lactic Acid with Polyglycolic Acid (PDLLA-co-PGA), Polyglycolic Acid with Trimethylene carbonate (PGA-co-TMC), Poly L Lactic Acid (PLLA) with β-Tricalcium phosphate (PLLA-TCP) and Poly L Lactic Acid (PLLA) with Hydroxylapatite (PLLA-HA) and combinations of these.

Additional Devices

The cement is required to set while tension is applied to the ligament. The ligament may therefore be attached to a device (maybe via sutures), which is itself attached to or supported by the bone. A set load could therefore be applied to the strands of the ligament, and these loads can be maintained while the cement sets.

I claim:

1. A prosthetic implant assembly for implantation in a bone joint between first and second bones, such as the knee joint between a femur and a tibia, said bone joint having bone tunnels formed in each of the first and second bones, and the implant assembly being intended to be introduced into the bone tunnels and to be fixated therein, in which the prosthetic implant assembly comprises:
a prosthetic ligament to be fitted in at least one of the bone tunnels; and,
a degradable adhesive and/or filler material (which may provide permanent or temporary fixation) to be introduced into said one bone tunnel to fill the space available between at least part of the prosthetic ligament and the wall of the bone tunnel, said material being of such a nature that (a) it is capable of setting in order temporarily to anchor said part of the ligament in position, and (b) it degrades over time such as to allow natural boney ingrowth to take over the anchoring of the prosthetic ligament.

2. A prosthetic implant kit for implantation in a bone tunnel in a bone joint, said kit comprising:
a prosthetic implant to be fitted in said bone tunnel;
a degradable adhesive and/or filler material to be introduced into said bone tunnel to fill the space available between at least part of the prosthetic implant and the wall of the bone tunnel, said material being of such a nature that (a) it is capable of setting in order temporarily to anchor said part of the implant in position, and (b) it degrades over time such as to allow natural boney ingrowth to take over the anchoring of the prosthetic implant; and,
a tunnel profiler to form an enlarged entry cavity to the bone tunnel to be filled with said material, and improve the temporary fixation of the implant.

3. A method of installing a prosthetic implant in a bone joint between first and second bones, such as the knee joint between a femur and a tibia, in which:
bone tunnels are formed in each of the first and second bones;
a prosthetic implant is taken through the bone tunnels; and,
a degradable adhesive and/or filler material is introduced into at least part of one of the tunnels and which is caused or allowed to set in order temporarily to anchor part of the implant in position, such material being of such a type as to degrade over time such as to allow natural boney ingrowth to take over the anchoring of the implant.

4. A prosthetic implant assembly according to claim 1, in which the degradable adhesive and/or filler material comprises a bone cement.

5. A prosthetic implant kit according to claim 2, in which the degradable adhesive and/or filler material is a bone cement.

6. A method according to claim 3, in which an enlarged entry cavity to a bone tunnel is formed by a tunnel profiler, and such cavity is filled with said material to improve the temporary fixation of the implant.

7. A method according to claim 6, in which a fixation device is provided to aid fixation between the implant and said material.
